(19) United States
(12) Patent Application Publication
(10) Pub. No.: US 2024/0315932 A1
(43) Pub. Date: Sep. 26, 2024

(54) Methods, Compositions and Devices Related to Seaweed Masks

(71) Applicant: Laclede, Inc., Rancho Dominguez, CA (US)

(72) Inventor: Michael Pellico, Rancho Dominguez, CA (US)

(21) Appl. No.: 18/439,657

(22) Filed: Feb. 12, 2024

Related U.S. Application Data
(63) Continuation of application No. 17/252,241, filed on Dec. 14, 2020, now abandoned.
(60) Provisional application No. 62/689,736, filed on Jun. 25, 2018.

(51) Int. Cl. A61K 8/02; A45D 44/00; A61K 8/04; A61K 8/25; A61K 8/34; A61K 8/73; A61K 8/9717; A61Q 19/00

(52) U.S. Cl. CPC A61K 8/0212; A45D 44/002; A61K 8/0216; A61K 8/042; A61K 8/25; A61K 8/345; A61K 8/735; A61K 8/9717; A61Q 19/00; A45D 2200/155; A61K 2800/48; A61K 2800/80

(57) ABSTRACT

Methods, compositions and devices for customizing a cosmetic mask for application to an individual, the cosmetic mask comprising a gel-like formulation that, when heated, can be customized for specific application to the individual. The invention further provides for a semi-solid starting formulation comprising a solvent, a humectant, a thickener, an acid, water and an effective amount of agar, wherein the semi-solid starting formulation forms a hydrogel when heated. Specifically, the semi-solid starting formulation, once heated, is applied to the skin of an individual while still warm, then forming a gel-like material once the formulation is allowed to fully cool on the skin.

### FIELD OF THE INVENTION

This application relates generally to the field of development and application of warm liquid face and body masks. Specifically, the relevant field includes methods, compositions and devices for making and using seaweed-based face and body masks for application to human subjects.

### BACKGROUND OF THE INVENTION

The benefits of using a gel-like mask for application to the face or body of an individual has been known in the art. In particular, these masks are generally pre-formed as a thin sheet in order to deliver specialized active agents to the skin, hair or other regions of an individual for the express purpose of improving certain health factors.

A variety of products in the cosmetic field have marketed such masks in order to promote wound healing aspects or to deliver active agents to the skin of an individual, including vitamins, moisturizers, conditions or similar compounds for the express intention of improving the appearance of the user.

While the pre-formed face mask has been known in the art, it has become only recently that natural product masks have entered the market in order to distinguish from synthetic or hydrogel masks. Known hydrogels in the state of the art are based on polyacrylic acids produced by polymerization (or salts thereof) or comprise further products that form gels by chemical synthesis, such as polyvinylpyrrolidone (PVP), polyvinyl alcohol (PVA) or other carbomers.

Natural products, on the other hand, are those masks developed from natural raw materials or natural polymers, including cellulose, gelatin, polysaccharides and mixtures thereof. Additionally, there are those natural products that come directly from raw materials, including seaweed- and kelp-based polysaccharides.

Unfortunately, pre-formed masks using natural products suffer from the inability of the natural products to form a firm enough gel that is sufficient to be applied to the skin of an individual in a manner that is intended to convey the desired benefit.

Thus, there remains a need in the art for developing products and devices that can generate a sufficiently firm mask based on natural and synthetically altered materials in order to be applied to the face or body of an individual, while utilizing a device that accommodates the formation and development of a personalized product that is user specific.

### SUMMARY OF THE INVENTION

The present invention provides for methods, compositions and devices for customizing a cosmetic mask for application to an individual, the cosmetic mask comprising a gel-like formulation that, when heated, can be customized for specific application to the individual.

In one aspect, the present invention describes devices that are specifically designed to heat a substance in a manner that oscillates between heating to a substance to near boiling then cooling to a temperature that allows for the substance to take the form of a gel-like mask for application to the face or body of an individual.

In another aspect, the present invention provides for a semi-solid starting formulation comprising a solvent, a humectant, a thickener, an acid, water and an effective amount of agar, wherein the semi-solid starting formulation forms a hydrogel when heated. Preferably, the semi-solid starting formulation, once heated, is applied to the skin of an individual while still warm, then forming a gel-like material once the formulation is allowed to fully cool on the skin.

These features, as well as various, alternative embodiments, will be apparent from a reading of the following detailed description of the present invention.

### BRIEF DESCRIPTION OF THE DRAWINGS

The novel features of the present invention are set forth herein embodied in the form of the claims of the invention. Features and advantages of the present invention may be best understood by reference to the following detailed description of the invention, setting forth illustrative embodiments and preferred features of the invention, as well as the accompanying drawings, of which:

FIG. 1 shows a container comprising the semi-solid formulation in prior to heating in the device of the present invention.

FIG. 2 shows the method steps of the present invention.

FIG. 3 shows the specialized heating apparatus for use in heating/cooling of the substance described in the present invention.

FIG. 4 shows the individual components of the apparatus and elements of the present invention.

### DETAILED DESCRIPTION OF THE INVENTION

Described herein are methods, compositions and devices for use in developing individualized face and body masks for users. The masks are formulated by use of a natural and synthetically altered product based substance that is applied after insertion into a specialized device. The device heats the substance to a near boiling temperature, after which time the substance is cooled sufficiently enough for application to the skin of an individual.

In some embodiments, the methods of the present invention further comprise the following steps: cleaning of the desired area for mask application; depositing semi-solid formulation in a thermo gel creation apparatus; allowing the thermo gel creation apparatus to heat the semi-solid formulation so that the semi-solid formulation is changed to a gel-like substance; applying the gel-like substance, once cooled into a mask, in layers to the face or body of an individual; allowing the mask to cool and firm in place for a period of time between 15 mins to 45 mins. Optionally, the period of time for the mask to cool and firm in place is between 10 mins to 40 mins.

In alternative embodiments, the devices of the present invention provide a heating element in order to bring the semi-solid formulation to a near boil temperature. Optionally, once the formulation is at near boil temperature, the formulation is then allowed to cool by removing the heat or adding a cooling element to the apparatus. Once the formulation is cooled sufficiently enough to avoid burning the skin of an individual, the formulation is brushed onto the face, body or skin of the individual, thereby forming a mask. Preferably, the mask is allowed to cool, while applied to the face or body, for a period of at least 10 mins to at least 40 mins. Most preferably, the cooling time is from 15 mins to 30 mins.

In certain embodiments, the natural and synthetically altered formulation is made using the following variations of materials:

Cellulose Derivatives: Examples of cellulose derivatives suitable for use herein include carboxymethyl hydroxyethylcellulose, carboxymethyl cellulose, carboxymethylcellulose sodium, cellulose acetate propionate carboxylate, hydroxyethylcellulose, hydroxyethyl ethylcellulose, hydroxypropylcellulose, methylcellulose, methylcellulose sodium, hydroxypropyl methylcellulose, methyl hydroxyethylcellulose, microcrystalline cellulose, sodium cellulose sulfate, and mixtures thereof. Also useful herein are the alkyl substituted celluloses. In these polymers, the hydroxy groups of the cellulose polymer is hydroyxalkylated (preferably hydroxyethylated or hydroxypropylated) to form a hydroxyalkylated cellulose which is then further modified with a C10-C30 straight chain or branched chain alkyl group through an ether linkage. Typically these polymers are ethers of C10-C30 straight or branched chain alcohols with hydroxyalkylcelluloses. Examples of alkyl groups useful herein include those selected from the group consisting of stearyl, isostearyl, lauryl, myristyl, cetyl, isocetyl, cocoyl (i.e. alkyl groups derived from the alcohols of coconut oil), palmityl, oleyl, linoleyl, linolenyl, ricinoleyl, behenyl, and mixtures thereof.

Natural Polymers: Suitable natural polymers for use herein include gelatin, polysaccharides, and mixtures thereof. Polysaccharides for use herein are preferably selected from red seaweed polysaccharides; glucomannans; galactomannans; fermentation polysaccharides, or derivatives thereof; brown seaweed polysaccharides; extracts of marine invertebrates; starch, or derivatives thereof; natural fruit extracts; plant fiber derivatives; kelp; natural plant exudates; resinous gums; and mixtures thereof. Preferably, locust bean gum is used as a thickening agent and viscosity enhancer for the semi-solid formulation of the present invention.

Gelatin: When gelatin is used in the devices herein, a high-molecular weight gelatin is combined with a low-molecular weight one to control the solubility. A gelatin having a low molecular weight of 20,000 or less is poor in gelling ability. Gelatin is an irreversibly hydrolived form of collagen wherein the hydrolysis reduces the protein fibrils into smaller peptides. This gelatin is otherwise known as hydrolyzed collagen. This preferred embodiment is used in the present invention as a gelling agent which will aid in skin elasticity and maintain moisture in an optimal manner.

Brown Seaweed Polysaccharides: Polysaccharides which are classified as brown seaweed polysaccharides are isolated by extraction from various species of Phaebophyceae. Suitable brown seaweed polysaccharides for use herein include algin, alginic acid, ammonium alginate, calcium alginate, potassium alginate, sodium alginate, propylene glycol alginate, and mixtures thereof.

Red Seaweed Polysaccharides: Polysaccharides which are classified as red seaweed polysaccharides are isolated from marine plant species belonging to the class of Rhodophyceae. Red seaweed polysaccharides provide mechanical strength to an aqueous gel. Preferably, the red seaweed polysaccharide for use herein is selected from agar, agarose, kappa-carrageenan and furcellaran, or mixtures thereof.

Glucomannan: Glucomannans are polysaccharides which comprise an essentially linear backbone of glucose and mannose residues. Glucomannans have short side branches attached to the linear backbone and acetyl groups are randomly present at the C-6 position of a sugar unit. The acetyl groups are generally found on one per six sugar units to one per twenty sugar units. Suitable glucomannans or derivatives thereof for use herein have a ratio of mannose to glucose of from about 0.2 to about 3. Preferred glucomannans for use herein include konjac mannan and deacetylated konjac mannan, or mixtures thereof.

Propanediol: Propanediol, or propylene glycol, is a synthetic organic compound that is viscous, colorless and used as a solvent in the present invention. It is also used as a preservative and may be derivable from kelp (propylene glycol alginate is an ester of alginic acid, which is derived from kelp). Propanediol or a compound having the structure:

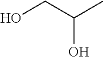

is a preferred embodiment of the solvent and/or preservative used in the present invention.

Agar: Agar, or agar-agar, is a jelly-like substance that is derived from red algae and has a structure of:

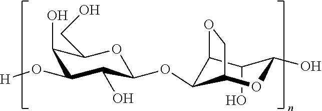

The chemical structure of agar consists of a mixture of two polysaccharides, agarose and agaropectin. Agarose, which comprises about 70% of the polysaccharide mixture, is a linear polymer comprising repeating units of agarobiose (a disaccharide consisting of D-galactose and 3,6-anhydro-L-galactopyranose). Agaropectin, comprising the other nearly 30% of the agar mixture, is an amalgamation of multiple smaller molecules that also includes repeating units of D-galactose and L-galactose. Agar is generally resistant to shear forces and exhibits a property known as hysteresis (Agar remains a gel at room temperature as high as 65° C., melting at approximately 85° C. while solidifying around 36° C.). Agar-agar is the preferred gelling agent of the present invention and is ideally suited for the semi-solid formulation to be stably heated, then become a gel-like substance when cooled.

Glycerin: The preferred humectant of the semi-solid formulation of the present invention, glycerin is a polyol compound having the structure:

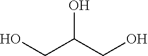

with the three hydroxyl groups conveying properties including solubility in water and hygroscopic nature of the compound. Glycerin may be synthetically produced or derived from plant sources, such as soybeans or palm. Glycerin provides a suitable lubricant property to the semi-solid formulation of the present invention.

Silica: Preferably, the formulations of the present invention utilize hydrophilic fumed silica (untreated fumed silica powder) in order to deliver certain enhanced properties as a thickener and an anti-settling agent. This form of silica also provides better adsorbancy rates and exhibits anti-caking of the formulation when mixed and applied to the skin of an individual.

Chlorophyllin-Copper Complex: Copper chlorophyllin (Cu-Chl) is a chlorophyll-derived mixture of different chlorin molecules known to have certain health benefits dues to its bioactive properties, such as anti-oxidant and deodorizing benefits. The preferred embodiment used herein is the copper derivative with a structure of:

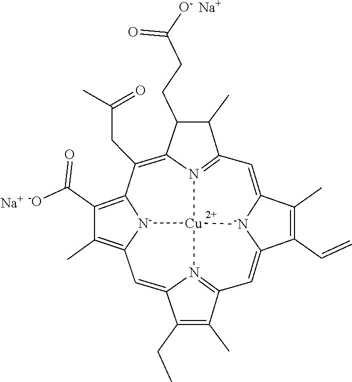

Fragrances: The semi-solid formulation of the present invention further comprises a selection of preferred fragrances with additional health benefits for use in the methods described herein. Specifically, certain oils and extracts are used, namely, oil derived from Boswellia carterii (Frankincense oil), extract of Pisum sativum (sweet pea oil) and Cucumis sativus extract (cucumber extract). Preferably, each oil or extract is present in an amount less than 2% of the total formulation.

Synthetic Peptides: Certain peptides have been shown to greatly improve skin conditioning and/or skin renewal based on the bioactive properties of the protein. The semi-solid formulations of the present invention preferably include synthetically derived epidermal growth factor as a skin conditioning agent.

Acids: Hyaluronic acid (HA) is naturally produced by the body, the largest amounts found in the skin, connective tissues and eyes. Its main function is to retain water and keep certain tissues moist/lubricated. HA is utilized in the present formulations to hydrate, repair and moisturize the skin as a part of the methods described herein.

Vitamins: Select vitamins that are important to the final form of the substance applied to the skin include vitamin E (tocopherol), vitamin C (ascorbic acid) and vitamin B₃ (niacinamide). These particulars vitamins are used herein for antioxidant and skin repair properties, protection from UV light and improve skin tones, respectively.

Activated charcoal: Optionally, the semi-solid formulation may include activated charcoal for extracting dirt and oil from pores within the skin.

### Examples

### I. Formulation Composition

A semi-solid formulation was developed based on a novel array of ingredients, comprising the following embodiments and in accordance with the following tables:

TABLE 1
Formulation A

| Ingredients | wt % range |
|---|---|
| Propanediol | 24-28% |
| Agar | 2-6% |
| Glycerin | 1-5% |
| Silica | 0.5-3% |
| Locust bean gum | 0.1-0.5% |
| Boswellia carteri oil | 0.02-0.1% |
| Pisum sativum oil | 0.02-0.1% |
| Cucumis sativus extract | 0.02-0.1% |
| Chlorophyll-copper complex | 0.009-0.03% |
| Hyaluronic acid | 0.01-.009% |
| Tocopherol | 0.01-.009% |
| Synthetic EGF | 0.001-0.005% |
| Ascorbic acid | 0.001-0.005% |
| Hydrolyzed collagen | 0.001-0.005% |
| Niacinamide | 0.001-0.005% |
| Purified water | 60-65% |

TABLE 2
Formulation B

| Ingredients | wt % range |
|---|---|
| Propanediol | 24-28% |
| Agar | 2-6% |
| Glycerin | 1-5% |
| Silica | 0.5-3% |
| Locust bean gum | 0.1-0.5% |
| Boswellia carteri oil | 0.02-0.1% |
| Pisum sativum oil | 0.02-0.1% |
| Cucumis sativus extract | 0.02-0.1% |
| Chlorophyll-copper complex | 0.009-0.03% |
| Hyaluronic acid | 0.01-.009% |
| Tocopherol | 0.01-.009% |
| Synthetic EGF | 0.001-0.005% |
| Ascorbic acid | 0.001-0.005% |
| Hydrolyzed collagen | 0.001-0.005% |
| Niacinamide | 0.001-0.005% |
| Activated charcoal | 0.1-0.4% |
| Purified water | 60-65% |

Formulations A and B are preferred embodiments for the compositions of the present invention. The main difference between the two Formulations is the addition of the activated charcoal element in Formulation B, which is to be used for oily skin.

These compositions were formulated using USP grade manufacturing techniques and stored in containers that did not allow for the semi-solid formulation to adhere to the inside.

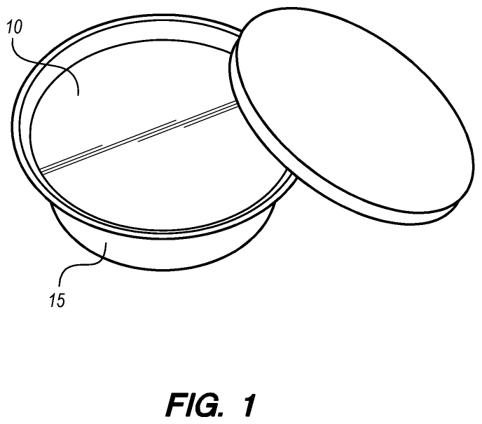

FIG. 1 shows a representative container 15 that maintains a stable form of the semi-solid formulation 10 of the present invention.

II. Use of Novel Heating Apparatus for Conversion of Semi-Solid Formulation to Liquid then Cooled for Application to Skin:

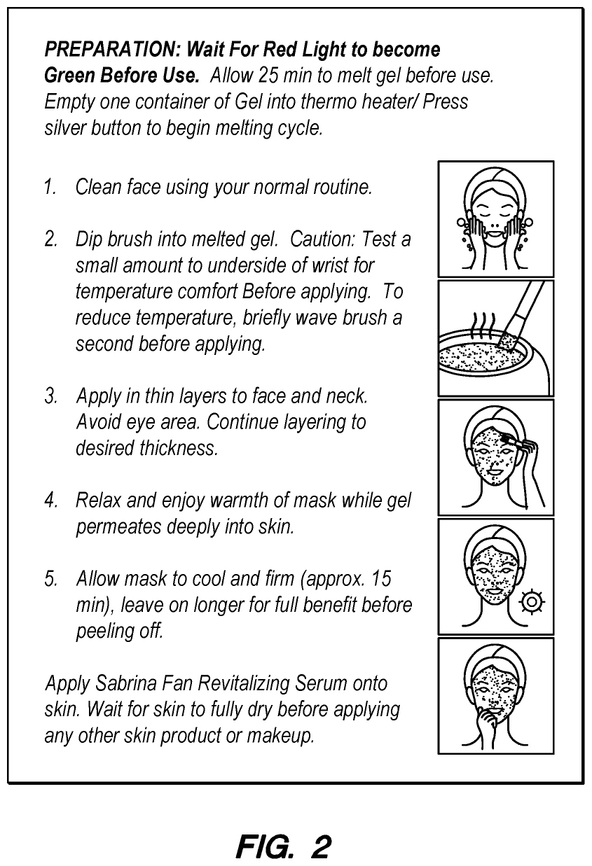

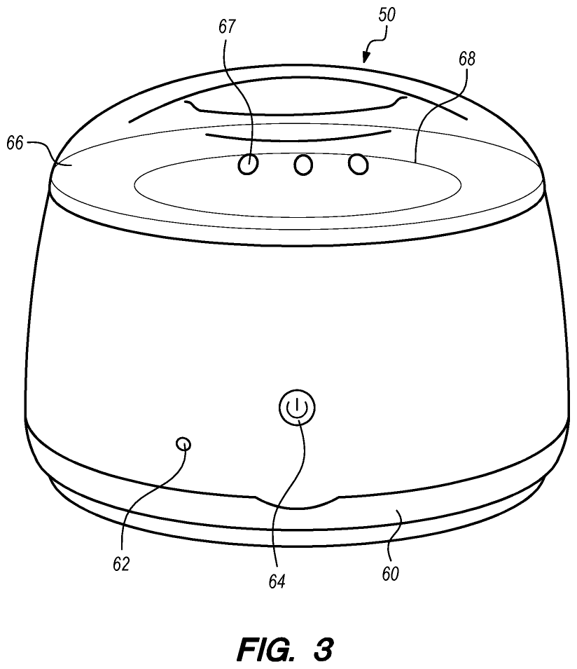

In accordance with exemplary method steps outlined at FIG. 2 and to appropriately apply the semi-solid formulation 10 to the skin of an individual, the formulation must be first heated (for approximately 25 minutes) to a temperature just under the boiling point of the formulation by the heating apparatus 50 in order to melt the semi-solid formulation 10 before use (FIG. 3). Optionally, the heating apparatus 50 will then lower the temperature (and maintain the lowered temperature) for a user to apply the formulation at the right time. This will enable the alteration of the semi-liquid formulation 10 to move from an initial form of a semi-solid that is then melted to a gel-like substance prior to application. Once the formulation has achieved the gel-like stage, the gel-like formulation is applied to the skin, where it will be appropriately cooled in order to form the final product of a thermal mask or cosmetic mask of the present invention.

Optionally, an applicator is used to apply the gel-like formulation to the skin of an individual.

FIG. 3 describes a preferred embodiment of the heating apparatus 50, which comprises an insulated housing 60 with an inner bowl (not shown) that can accommodate a smaller, removable basket 68 in which the semi-solid formulation 10 is initially deposited prior to melting.

Heating apparatus 50 may also comprise power button 64 and indicator light 62 that displays different colors depending on the temperature of the formulation during the heating stage. Lid 66 is placed atop the heating apparatus 50 with vents 67 in order to maintain appropriate temperatures throughout heating.

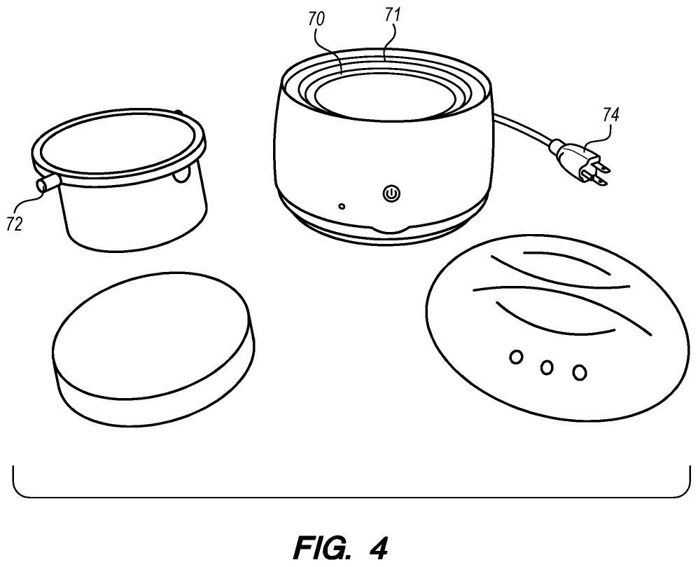

As shown at FIG. 4, the device of the present invention is divided into component parts. Heating apparatus 50 has a voltage of 110V-220V at 60 Hz, with a total power of 150 W, whereby power cord 74 connect to an appropriate AC outlet. This power supply allows for inner bowl 70 to heat removable basket 68 to the optimal temperature to liquefy the initial, semi-solid formulation 10 to a temperature just below the boiling point. Removable basket 68 may be removed from heating apparatus 50 by way of handle 72 in order to protect user from heat. Gasket 71 is provided to maintain temperature and provide insulation to housing 60.

The present invention provides improvements over the existing state of the art for the following reasons. Firstly, the current products on the market are either pre-formed masks or are liquids that are applied cold. This actually closes the skin pores, thereby preventing deeper penetration of the substances of the present invention into the skin, resulting in a loss of efficacy and therapeutic value to the user. Secondly, the existing products on the market are water based and require long drying times on the skin of up to an hour (depending on the applied level of thickness). The present invention is applied warm to cool in temperature, using a gel-like material that can be removed in a matter of minutes without losing the desired efficacy. Furthermore, the gel-like mask provides the benefits of an initial warm application (thus opening the skin pores of a user) with the additional benefits of keeping the mask in place as it cools (thereby making it easier to remove from the face or body by peeling off, leaving little to no residue when compared to a liquid form).

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features. As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should, therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

What is claimed is:

1. A formulation for use as a cosmetic mask, the formulation comprising:
(a) a solvent,
(b) a humectant,
(c) a thickener,
(d) an acid,
(e) an amount of purified water; and
(f) an effective amount of agar, wherein the semi-solid starting formulation forms a hydrogel when heated.

2. The formulation of claim 1, wherein the semi-solid starting formulation, once heated, is applied to the skin of an individual while still warm, then forming a gel-like material once the formulation is allowed to fully cool on the skin.

3. The formulation of claim 1, wherein the solvent is propanediol.

4. The formulation of claim 1, wherein the humectant is glycerin.

5. The formulation of claim 1, wherein the thickener is silica.

6. The formulation of claim 1, wherein the acid is hyaluronic acid.

7. The formulation of claim 1, wherein the amount of purified water is present in an amount that is greater than 50 wt %.

8. The formulation of claim 1, wherein the agar is derived from Gelidium cartilagineum.

9. The formulation of claim 1, further comprising at least one oil or extract selected from the group consisting of Boswellia carterii, Pisum sativum and Cucumis sativus.

10. The formulation of claim 9, wherein the amount of the at least one oil or extract totals no more than 2% of the total formulation.

11. The formulation of claim 1, further comprising a viscosity enhancer comprising an amount of locust bean gum.

12. The formulation of claim 1, further comprising an effective amount of chlorophyllin-copper complex.

13. The formulation of claim 1, further comprising a skin conditioner, wherein the skin conditioner is a synthetic form of epidermal growth factor.

14. The formulation of claim 1, further comprising at least two vitamins selected from the group consisting of vitamin E, vitamin C and vitamin B₃.

15. The formulation of claim 1, further comprising an amount under 2 wt % of hydrolyzed collagen.

16. The formulation of claim 1, further comprising an effective amount of activated charcoal.

17. A method of treating a portion of the skin of an individual, comprising:
(a) providing an amount of the formulation of claim 1;
(b) providing a heating apparatus to heat the formulation of (a);
(c) allowing the formulation to liquefy; and
(d) allowing the liquefied formulation to cool prior to applying the formulation to the skin of an individual.
